(19) United States
(12) Patent Application Publication
(10) Pub. No.: US 2023/0380712 A1
(43) Pub. Date: Nov. 30, 2023

(54) APPARATUS FOR NANOPARTICLE THERMAL THERAPY BASED ON OPEN MAGNETIC PARTICLE IMAGE

(71) Applicant: GIST(Gwangju Institute of Science and Technology), Gwangju (KR)

(72) Inventors: Jung Won YOON, Gwangju (KR); Tuan Anh LE, Gwangju (KR); Minh Phu BUI, Gwangju (KR)

(73) Assignee: GIST(Gwangju Institute of Science and Technology), Gwangju (KR)

(21) Appl. No.: 18/446,152

(22) Filed: Aug. 8, 2023

Related U.S. Application Data
(63) Continuation of application No. PCT/KR2021/019961, filed on Dec. 27, 2021.

(30) Foreign Application Priority Data
Feb. 8, 2021 (KR) 10-2021-0017927

(51) Int. Cl. A61B 5/0515; A61N 2/02; A61K 41/00

(52) U.S. Cl. CPC A61B 5/0515; A61N 2/02; A61K 41/0052

(57) ABSTRACT

The present invention relates to apparatus for a nanoparticle thermal therapy based on an open magnetic particle image. The apparatus for a nanoparticle thermal therapy based on an open magnetic particle image according to an exemplary embodiment of the present invention may include: a selection coil generating a field free point (FFP) for a nanoparticle in a first direction; a focus coil generating a magnetic field and moving the FFP in a second direction; and a heating coil heating the nanoparticle in a target area based on the FFP in the second direction.

### CROSS-REFERENCE TO RELATED APPLICATIONS

This application is a Continuation of International Application No. PCT/KR2021/019961 filed on Dec. 27, 2021, which claims the priority of Korean Patent Application No. 10-2021-0017927 filed on Feb. 8, 2021, in the Korean Intellectual Property Office, the disclosure of which is incorporated herein by reference.

### BACKGROUND OF THE INVENTION

### Field of the Invention

The present invention relates to an apparatus for a nanoparticle thermal therapy based on an open magnetic particle image, and more particularly, to an apparatus for a nanoparticle thermal therapy based on an open magnetic particle image for a targeted therapy.

### Description of the Related Art

A thermal therapy is a disease therapy method using characteristics in which cancer cells are weak to heat and die at a temperature of 42° C. or higher. Currently, hospitals use microwaves, infrared rays, ultrasonic waves, and lasers as means of generating heat, but in this case, in that heat cannot penetrate deep into the body and due to a limit that local heating of only the diseased area is difficult, which can damage normal tissues, the means is used only as an adjunct to drug treatment.

In order to overcome these disadvantages, a new concept of self-thermal therapy that combines recent nanotechnology with the existing thermal therapy method has been proposed, but research on this is insufficient.

### SUMMARY OF THE INVENTION

The present invention is contrived to solve the problem, and an object of the present invention is to provide an apparatus for a nanoparticle thermal therapy based on an open magnetic particle image.

Further, an object of then present invention is to provide an apparatus for heating the nanoparticle in a target area based on an FFP.

The objects of the present invention are not limited to the aforementioned objects, and other objects, which are not mentioned above, will be apparent from the following description.

In order to achieve the objects, an apparatus for a nanoparticle thermal therapy based on an open magnetic particle image according to an exemplary embodiment of the present invention may include: a selection coil generating a field free point (FFP) for a nanoparticle in a first direction; a focus coil generating a magnetic field and moving the FFP in a second direction; and a heating coil heating the nanoparticle in a target area based on the FFP in the second direction.

In the exemplary embodiment, the selection coil may include a lower selection coil and an upper selection coil positioned on the lower selection coil, and the lower selection coil and the upper selection coil may be positioned with opposite magnetisms.

In the exemplary embodiment, the apparatus for a nanoparticle thermal therapy based on an open magnetic particle image may further include: a first excitation coil for oscillating the nanoparticle; and a receiver coil for detecting a signal for the nanoparticle.

In the exemplary embodiment, the apparatus for a nanoparticle thermal therapy based on an open magnetic particle image may further include: a second excitation coil generating an induced voltage having an opposite polarity to the induced voltage generated by the first excitation coil; and an attenuation coil configured in a winding direction which is opposite to a winding direction of the receiver coil.

In the exemplary embodiment, a received signal of the receiver coil may be used for measuring the temperature of the nanoparticle.

In the exemplary embodiment, the apparatus for a nanoparticle thermal therapy based on an open magnetic particle image may further include a stage unit for adjusting a position of the target area corresponding to the nanoparticle.

In the exemplary embodiment, the nanoparticle may be heated in a first time interval and the temperature of the nanoparticle may be measured in a second time interval, and a third time interval between the first time interval and the second time interval may include a relaxation time.

Specific details for achieving the above objects will become clear with reference to embodiments to be described later in detail in conjunction with the accompanying drawings.

However, the present invention is not limited to an exemplary embodiment disclosed below but may be implemented in various different shapes and the present embodiment just completes a disclosure of the present invention and is provided to completely inform a scope of the present invention to those skilled in the art to which the present invention belongs (hereinafter, referred to as “those skilled in the art”).

According to an exemplary embodiment of the present invention, targeted thermal therapy capable of generating high-efficiency heat generation can be performed while minimizing side effects with biocompatible magnetic nanoparticles.

In addition, according to an exemplary embodiment of the present invention, image feedback can be performed through a body scale open magnetic particle imaging (MPI) based magnetic particle temperature image measurement for a user.

Further, according to an exemplary embodiment of the present invention, image-based feedback for targeting cancer treatment sites with magnetic nanoparticles and temperature feedback of nanoparticles can be performed.

The effects of the present invention are limited to the above-described effects, and the potential effects expected by the technical features of the present invention will be clearly understood from the description below.

### BRIEF DESCRIPTION OF THE DRAWINGS

FIGS. 1A to 1D are diagrams illustrating an apparatus for a nanoparticle thermal therapy based on an open magnetic particle image according to an exemplary embodiment of the present invention.

FIGS. 2A and 2B are diagrams illustrating a magnetic particle imaging unit according to an exemplary embodiment of the present invention.

FIG. 3 is a diagram illustrating a heating and temperature measurement graph according to an exemplary embodiment of the present invention.

FIG. 4 is a diagram illustrating a selection coil according to an exemplary embodiment of the present invention.

FIG. 5 is a diagram illustrating a focus coil according to an exemplary embodiment of the present invention.

FIG. 6 is a diagram illustrating a heating coil according to an exemplary embodiment of the present invention.

FIG. 7 is a diagram illustrating an example of a thermal therapy according to an exemplary embodiment of the present invention.

FIGS. 8A and 8B are diagrams illustrating signal detection for where there is a nanoparticle according to an exemplary embodiment of the present invention.

FIG. 9 is a diagram illustrating a receiver coil and an attenuation coil according to an exemplary embodiment of the present invention.

FIGS. 10A and 10B are diagrams illustrating a system for heating a nanoparticle based on an open magnetic particle image according to an exemplary embodiment of the present invention.

### DETAILED DESCRIPTION OF THE PREFERRED EMBODIMENT

The present invention may have various modifications and various exemplary embodiments and specific exemplary embodiments will be illustrated in the drawings and described in detail.

Various features of the invention disclosed in the claims may be better understood in consideration of the drawings and detailed description. Devices, methods, manufacturing methods, and various embodiments disclosed in the specification are provided for illustrative purposes. The disclosed structural and functional features are intended to enable a person skilled in the art to specifically implement various embodiments, and are not intended to limit the scope of the invention. The disclosed terms and phrases are intended to provide an easy-to-understand description of the various features of the disclosed invention, and are not intended to limit the scope of the invention.

In describing the present invention, a detailed description of related known technologies will be omitted if it is determined that they unnecessarily make the gist of the present invention unclear.

Hereinafter, an apparatus for a nanoparticle thermal therapy based on an open magnetic particle image according to an exemplary embodiment of the present invention will be described.

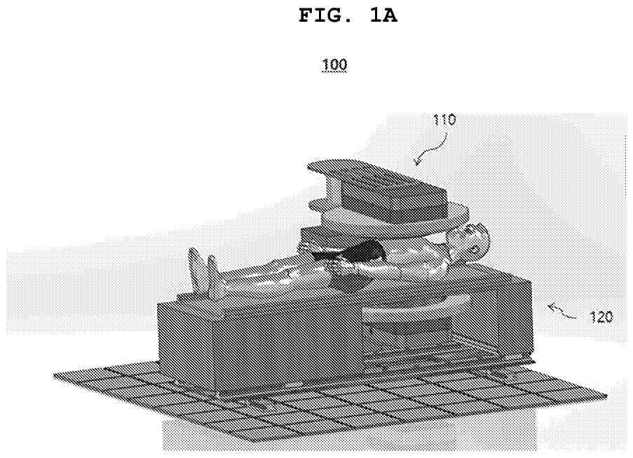

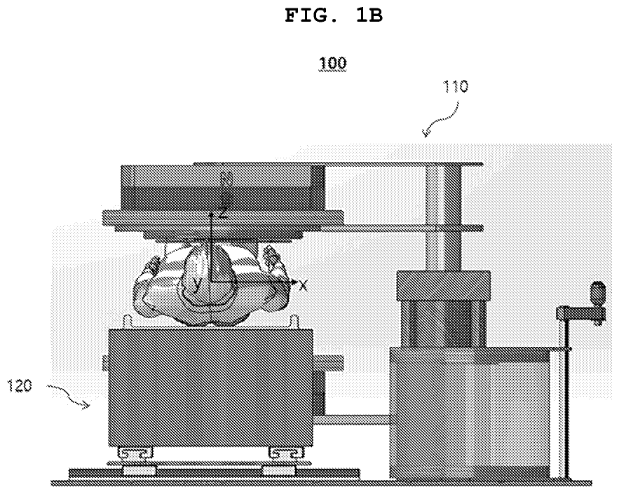

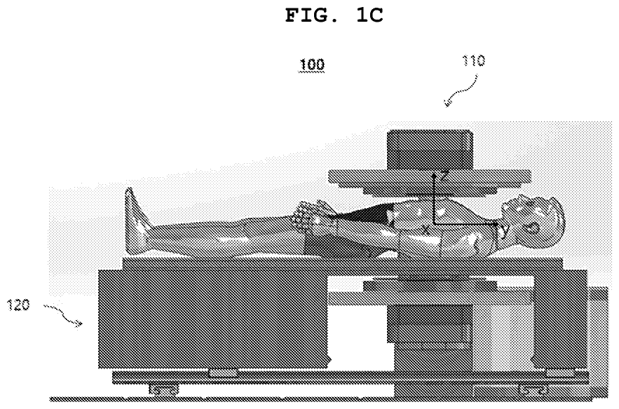

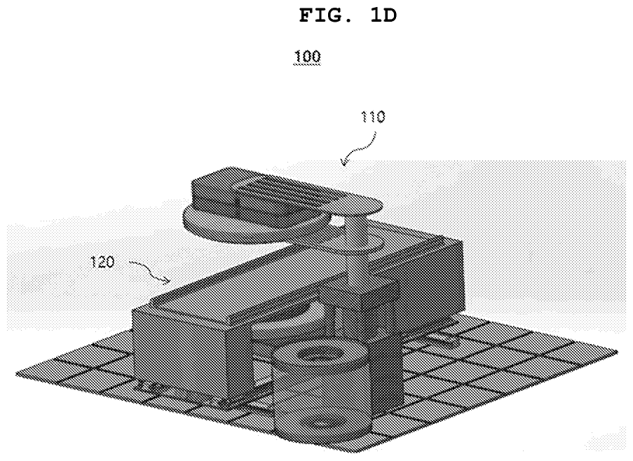

FIGS. 1A to 1D are diagrams illustrating an apparatus 100 for a nanoparticle thermal therapy based on an open magnetic particle image according to an exemplary embodiment of the present invention.

Referring to FIGS. 1A to 1D, the nanoparticle thermal therapy apparatus 100 may include a magnetic particle imaging unit 110 and a storage unit 120.

The magnetic particle imaging unit 110 a targeted thermal therapy capable of generating high-efficiency heat generation while minimizing side effects with biocompatible magnetic nanoparticles.

In addition, the magnetic particle imaging unit 110 may perform image feedback through a body scale open magnetic particle imaging (MPI) based magnetic particle temperature image measurement for a user.

For example, the magnetic particle imaging unit 110 may perform image-based feedback for targeting cancer treatment sites with magnetic nanoparticles and temperature feedback of nanoparticles.

In an exemplary embodiment, the magnetic particle imaging unit 110 is configured in an open type to exponentially reduce the user's risk of claustrophobia and panic attacks and may accurately diagnose problems for users of all shapes and sizes.

Further, the magnetic particle imaging unit 110 may be combined with another system such as an operation system controlling the nanoparticle.

In addition, the magnetic particle imaging unit 110 may obtain a 3D MPI image at a low speed. For example, one may electronically indicate an FFP scan direction and a second one may mechanically indicate an FFP scan direction

In an exemplary embodiment, instead of using a directly heating frequency (100 kHz-1 MHz), an excitation signal (low frequency of 10 kHz-30 kHz) is added, so a smaller bandwidth may be required for a channel receive signal during temperature measurement.

The stage unit 120 may adjust a position of the target area of the magnetic particle imaging unit 110 corresponding to the nanoparticle.

In an exemplary embodiment, the stage unit 120 may include a fixing member for seating the user, and move the fixing member in a horizontal direction (x-axis, y-axis) in order to a therapy position of the magnetic particle imaging unit 110 for the user.

Further, the stage unit 120 may adjust the magnetic particle imaging unit 110 in a height direction (z-axis) to adjust a separation distance of the magnetic particle imaging unit 110 from the user.

Referring to FIGS. 1A to 1D, in the apparatus 100 for a nanoparticle thermal therapy based on an open magnetic particle image in various embodiments of the present invention, the components described in FIGS. 1A to 1D are not essential, so the apparatus 100 may be implemented to have more components than the components described in FIGS. 1A to 1D or to have less components therethan.

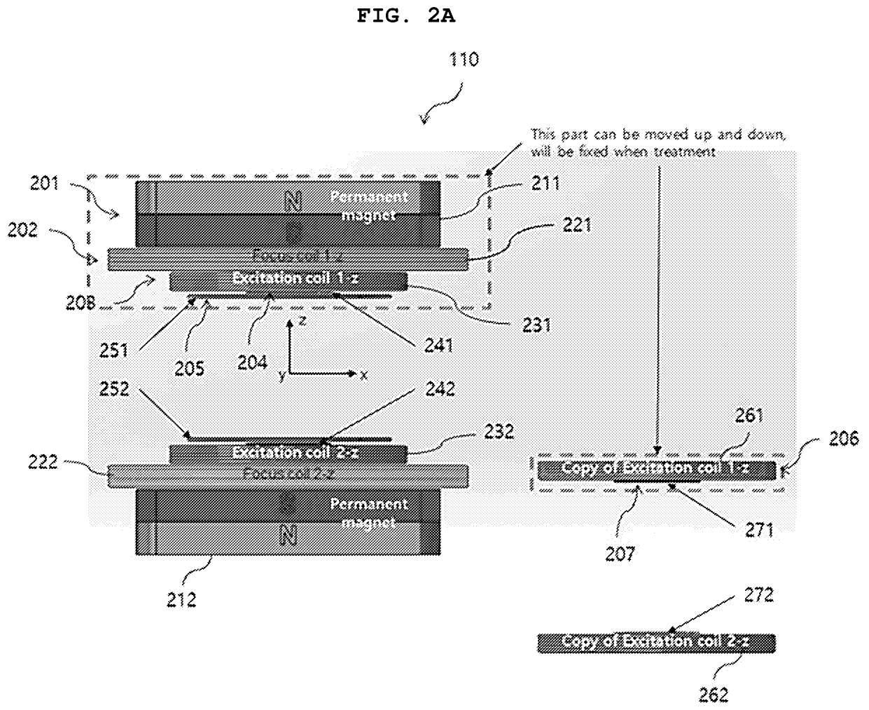

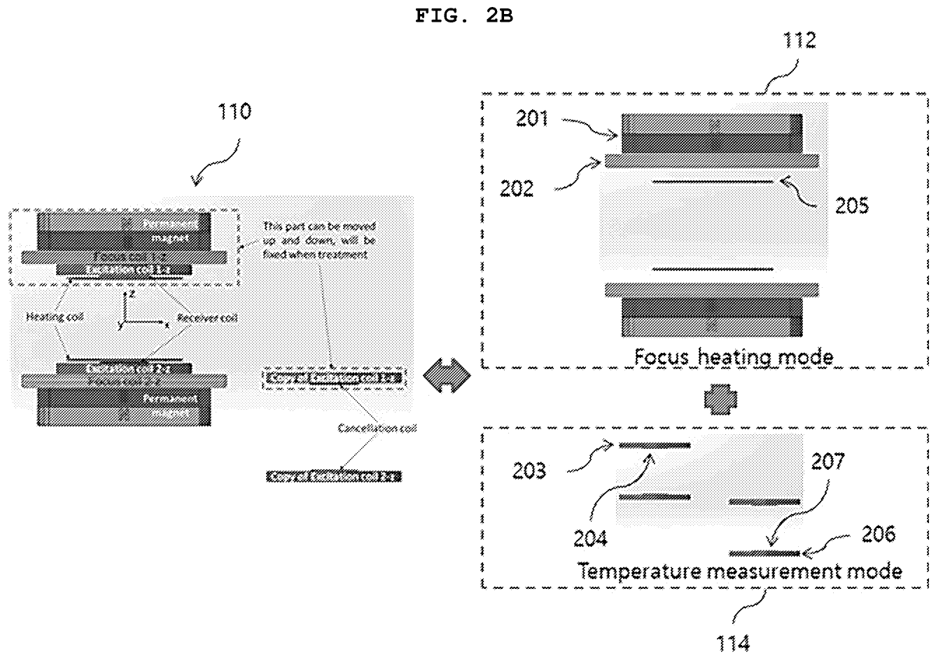

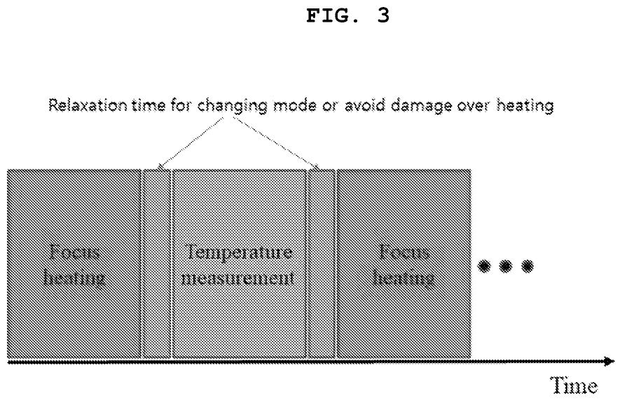

FIGS. 2A and 2B are diagrams illustrating a magnetic particle imaging unit 110 according to an exemplary embodiment of the present invention. FIG. 3 is a diagram illustrating a heating and temperature measurement graph according to an exemplary embodiment of the present invention.

Referring to FIGS. 2A and 2B, the magnetic particle imaging unit 110 may include a target heating coil 112 and a temperature measuring coil 114.

The target heating coil 112 may perform the thermal therapy by heating the nanoparticle in the target area. The temperature measuring coil 114 may control a heated temperature. For example, referring to FIG. 3, the nanoparticle is heated in a first time interval (focus heating), the temperature of the nanoparticle is measured in a second time interval (temperature measurement), and a third time interval between the first time interval and the second time interval may include a relaxation time, and in this case, damage through mode switching or heating may be suppressed.

In an exemplary embodiment, the target heating coil 112 may include a selection coil 201, a focus coil 202, and a heating coil 205.

In an exemplary embodiment, the selection coil 201 may generate a field free point (FFP) for the nanoparticle in a first direction.

In an exemplary embodiment, the focus coil 202 generates a magnetic field to move the FFP in a second direction.

In an exemplary embodiment, the heating coil 205 may heat the nanoparticle in the target area based on the FFP in the second direction.

In an exemplary embodiment, the temperature measuring coil 114 may include a first excitation coil 203, a receiver coil 204, a second excitation coil 206, and an attenuation coil 207.

In an exemplary embodiment, the first excitation coil 203 may oscillate the nanoparticle.

In an exemplary embodiment, the receiver coil 204 may detect a signal for the nanoparticle. In an exemplary embodiment, the received signal of the receiver coil 204 may be used to measure the temperature of the nanoparticle.

In an exemplary embodiment, the second excitation coil 206 may generate an induced voltage an induced voltage having an opposite polarity to the induced voltage generated by the first excitation coil 203.

In an exemplary embodiment, the attenuation coil 207 may be configured in a winding direction opposite to that of the receiver coil 204.

In an exemplary embodiment, the selection coil 201 may include a lower selection coil 212 and an upper selection coil 211 positioned on the lower selection coil 212.

In an exemplary embodiment, the focus coil 202 may include a lower focus coil 222 and an upper focus coil 221 positioned on the lower focus coil 222.

In an exemplary embodiment, the first excitation coil 203 may include a first lower excitation coil 232 and a first upper excitation coil 231 positioned on the first lower excitation coil 232.

In an exemplary embodiment, the receiver coil 204 may include a lower receiver coil 242 and an upper receiver coil 241 positioned on the lower receiver coil 242.

In an exemplary embodiment, the heating coil 205 may include a lower heating coil 252 and an upper heating coil 251 positioned on the lower heating coil 252.

In an exemplary embodiment, the second excitation coil 206 may include a second lower excitation coil 262 and a second upper excitation coil 261 positioned on the second lower excitation coil 262.

In an exemplary embodiment, the attenuation coil 207 may include a lower attenuation coil 272 and an upper attenuation coil 271 positioned on the lower attenuation coil 272.

In an exemplary embodiment, the lower focus coil 222 may be positioned on the lower selection coil 212, the first lower excitation coil 232 may be positioned on the lower focus coil 222, the lower receiver coil 242 may be positioned on the first lower excitation coil 232, and the lower heating coil 242 may be positioned on the lower receiver coil 242.

In an exemplary embodiment, the upper focus coil 251 may be positioned on the lower heating coil 252, the upper receiver coil 241 may be positioned on the upper heating coil 251, the first upper excitation coil 231 may be positioned on the upper receiver coil 241, the upper focus coil 221 may be positioned on the first upper excitation coil 231, and the upper selection coil 211 may be positioned on the upper focus coil 221.

In an exemplary embodiment, a field of view (FOV) may be formed between the lower heating coil 252 and the upper heating coil 251. In this case, a nanoparticle corresponding to the user may be positioned inside the FOV.

In an exemplary embodiment, the lower attenuation coil 272 may be positioned on the second lower excitation coil 262, the upper attenuation coil 271 may be positioned on the lower attenuation coil 272, and the second upper excitation coil 261 may be positioned on the upper attenuation coil 271.

In this case, the second lower excitation coil 262, the lower attenuation coil 272, the upper attenuation coil 271, and the second upper excitation coil 261 may be positioned on the side of the first lower excitation coil 232, the lower receiver coil 242, the upper receiver coil 241, and the first upper excitation coil 231, and heights may be adjusted in a height direction (z axis).

Referring to FIGS. 2A and 2B, in various embodiments of the present invention, the magnetic particle imaging unit 110 may be implemented to have more components than the components described in FIGS. 2A and 2B or to have less components therethan because the components described in FIGS. 2A and 2B are not required.

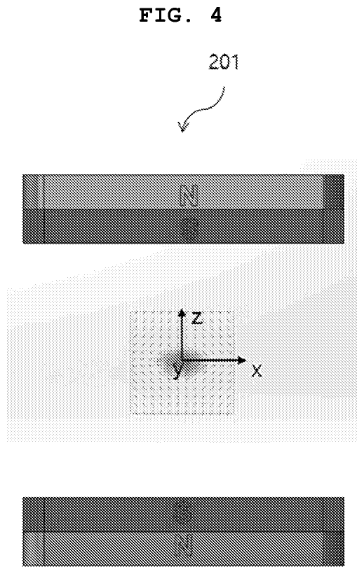

FIG. 4 is a diagram illustrating a selection coil 201 according to an exemplary embodiment of the present invention.

Referring to FIG. 4, the selection coil 201 may be configured by a permanent magnet for generating the FFP at a center.

In an exemplary embodiment, the lower selection coil 212 and the upper selection coil 211 of the selection coil 201 may be positioned with opposite magnetisms.

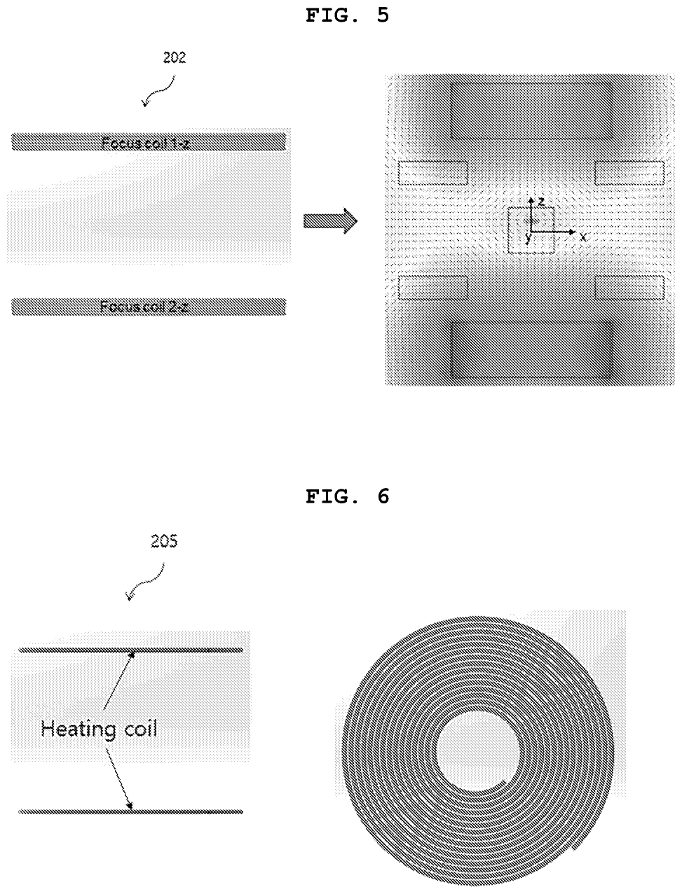

FIG. 5 is a diagram illustrating a focus coil 202 according to an exemplary embodiment of the present invention.

Referring to FIG. 5, the focus coil 202 may generate a uniform magnetic field to move the FFP in the z-axis of the target area.

In an exemplary embodiment, the focus coil 202 may include a Helmholz configuration.

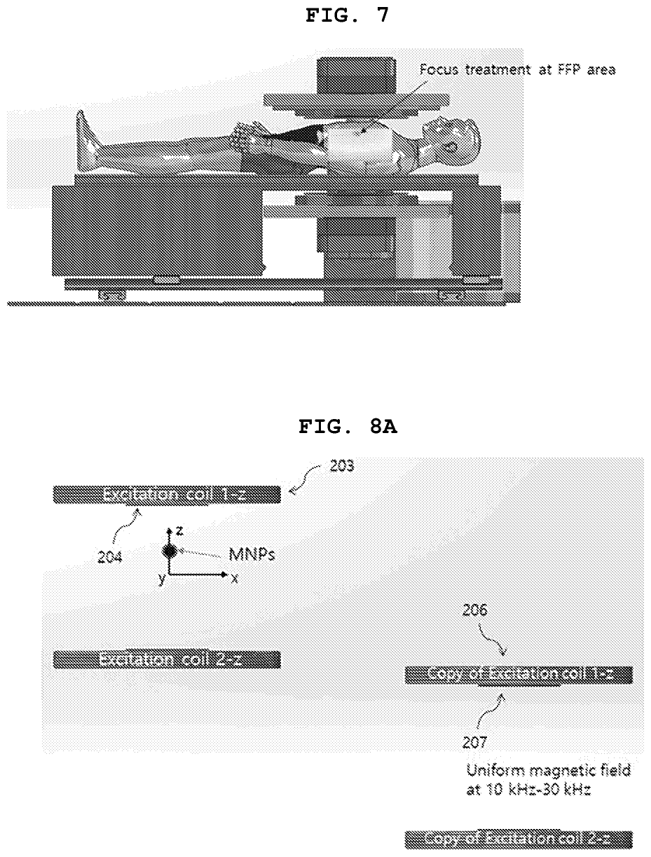

FIG. 6 is a diagram illustrating a heating coil 205 according to an exemplary embodiment of the present invention. FIG. 7 is a diagram illustrating an example of a thermal therapy according to an exemplary embodiment of the present invention.

Referring to FIG. 6, the heating coil 205 may heat the nanoparticles for the thermal therapy. For example, the heating coil 205 may use a water cooling scheme.

In one embodiment, the heating coil 205 may be configured as a spiral coil to generate a uniform heating field in the target area.

In this case, a heating field frequency may include 100 kHz-1 Mhz, f*H<=5×10⁹ A/m/s.

Referring to FIG. 7, the target area may be matched with the FFP by mechanically moving the fixing member of the stage on which the user is seated. For example, the fixed member may move in a two-dimensional plane of the x-axis and the t-axis through a rail configuration.

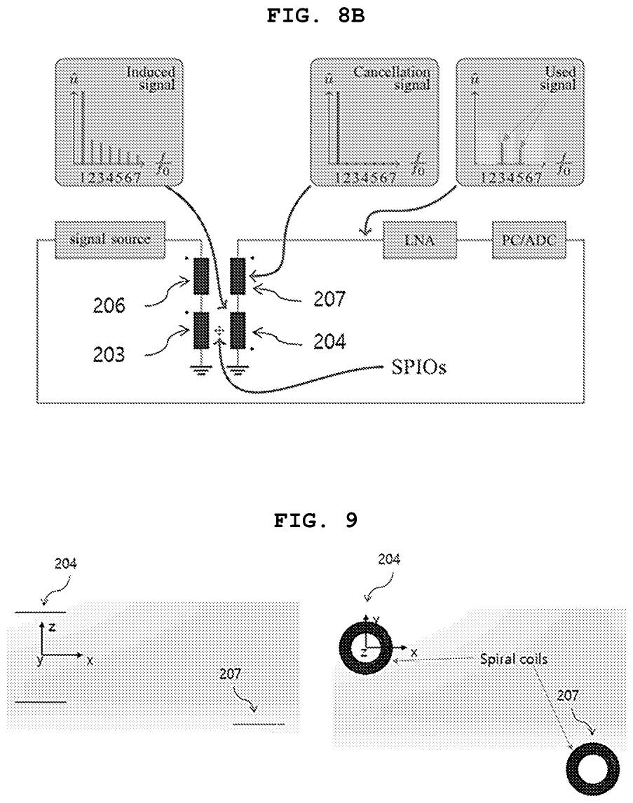

FIGS. 8A and 8B are diagrams illustrating signal detection for where there is a nanoparticle according to an exemplary embodiment of the present invention.

Referring to FIGS. 8A and 8B, the winding direction of the receiver coil 204 and the winding direction of the attenuation coil 207 may be opposite to each other. In this case, the receiver coil 204 and the attenuation coil 207 may be used to measure the temperature of the nanoparticle (MNP).

In an exemplary embodiment, a receiver signal of the receiver coil 204 may be expressed as in <Equation 1> below.

u(t)=u_{E}(t)+u_{C}(t)+u_{P}(t)˜u_{P}(t) [Equation 1]

Here, u(t) represents the receiver signal, uE(t) represents the induced voltage generated by the first excitation coil unit 203, uC(t) represents the induced voltage generated by the attenuation coil unit 207, and uP (t) represents the signal for the nanoparticle.

In an exemplary embodiment, the induced voltage generated by the first excitation coil 203 and the induced voltage generated by the attenuation coil 207 may be expressed as <Equation 2> below.

u_{C}(t)=−u_{E}(t) [Equation 2]

In an exemplary embodiment, the first excitation coil unit 203 may include a Helmholz configuration, and the first excitation coil unit 203 may be configured in the same form as the second excitation coil unit 206.

In an exemplary embodiment, a third harmonic and a fifth harmonic may be obtained for use in temperature measurement.

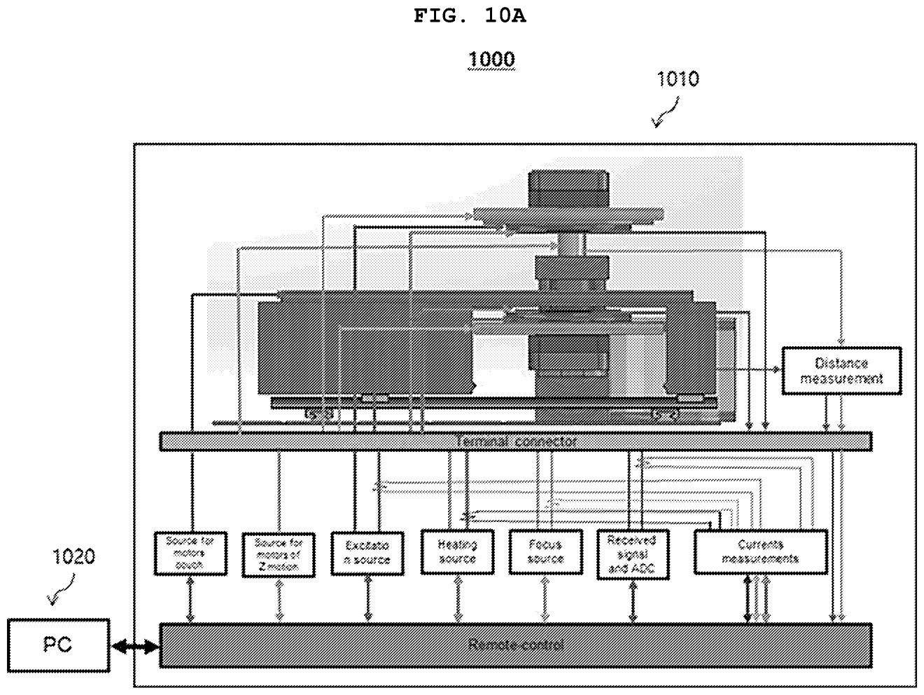

FIG. 9 is a diagram illustrating a receiver coil 204 and an attenuation coil 207 according to an exemplary embodiment of the present invention.

Referring to FIG. 9, the receiver coil 204 and the attenuation coil 207 may have the same structure, and in this case, winding directions may be configured opposite to each other.

In an exemplary embodiment, the receiver coil 204 and the attenuation coil 207 may be configured by spiral coils.

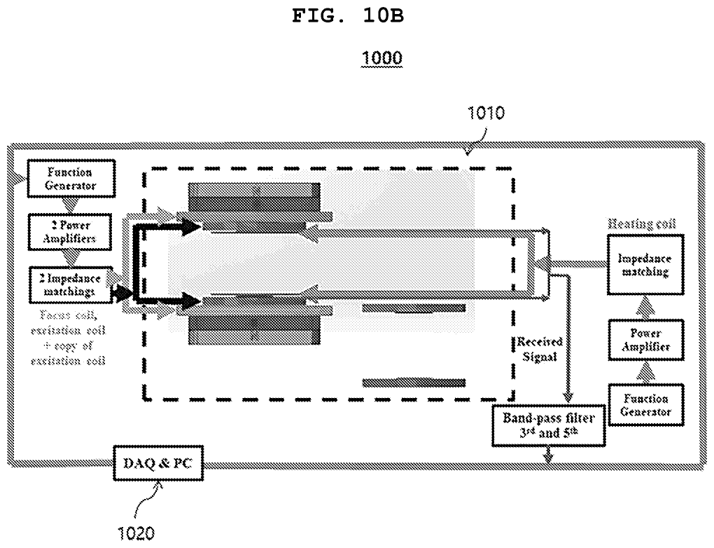

FIGS. 10A and 10B are diagrams illustrating a system 1000 for heating a nanoparticle based on an open magnetic particle image according to an exemplary embodiment of the present invention.

Referring to FIGS. 10A and 10B, the nanoparticle heating system 1000 may include a nanoparticle thermal therapy apparatus 1010 and an external electronic apparatus 1020.

For example, the external electronic apparatus 1020 may include various terminal devices such as a PC, a laptop computer, a tablet PC, and a smart phone.

In an exemplary embodiment, the nanoparticle thermal therapy apparatus 1010 may include a nanoparticle thermal therapy apparatus 100 of FIGS. 1A to 1D, a communication unit (not illustrated), and a control unit (not illustrated).

The communication unit may transmit a magnetic particle image to the external electronic apparatus 1020 so that the magnetic particle image is displayed by the external electronic apparatus 1020.

In an exemplary embodiment, the communication unit may include at least one of a wired communication module and a wireless communication module. A part or the entirety of the communication unit may be referred to as ‘transmitter’, ‘receiver’, or ‘transceiver’.

The control unit may measure the temperature of the nanoparticle. In addition, the control unit may control the stage unit of the nanoparticle thermal therapy apparatus 100 to adjust the position of the user and the position of the coil.

In an exemplary embodiment, the control unit may include at least one processor or micro processor, or may be part of the processor. In addition, the control unit may be referred to as a communication processor (CP). The control unit may control an operation of the nanoparticle heating system 1000 according to various embodiments of the present invention.

Referring to FIGS. 10A and 10B, in various embodiments of the present invention, the system 1000 for heating a nanoparticle based on an open magnetic particle image may be implemented to have more components than the components described in FIGS. 10A and 10B or to have less components therethan because the components described in FIGS. 10A and 10B are not required.

The above description just illustrates the technical spirit of the present invention and various changes and modifications can be made by those skilled in the art to which the present invention pertains without departing from an essential characteristic of the present invention.

The various embodiments disclosed herein may be performed in any order, simultaneously or separately.

In an exemplary embodiment, at least one step may be omitted or added in each figure described in this specification, may be performed in reverse order, or may be performed simultaneously.

The exemplary embodiments of the present invention are provided for illustrative purposes only but not intended to limit the technical spirit of the present invention. The scope of the present invention is not limited to the exemplary embodiments.

The protection scope of the present invention should be construed based on the following appended claims and it should be appreciated that the technical spirit included within the scope equivalent to the claims belongs to the scope of the present invention.

What is claimed is:

1. An apparatus for a nanoparticle thermal therapy based on an open magnetic particle image, the apparatus comprising:
a selection coil generating a field free point (FFP) for a nanoparticle in a first direction;
a focus coil generating a magnetic field and moving the FFP in a second direction; and
a heating coil heating the nanoparticle in a target area based on the FFP in the second direction.

2. The apparatus for a nanoparticle thermal therapy based on an open magnetic particle image of claim 1, wherein the selection coil includes a lower selection coil and an upper selection coil positioned on the lower selection coil, and
the lower selection coil and the upper selection coil are positioned with opposite magnetisms.

3. The apparatus for a nanoparticle thermal therapy based on an open magnetic particle image of claim 1, further comprising:
a first excitation coil for oscillating the nanoparticle; and
a receiver coil for detecting a signal for the nanoparticle.

4. The apparatus for a nanoparticle thermal therapy based on an open magnetic particle image of claim 3, further comprising:
a second excitation coil generating an induced voltage having an opposite polarity to the induced voltage generated by the first excitation coil; and
an attenuation coil configured in a winding direction which is opposite to a winding direction of the receiver coil.

5. The apparatus for a nanoparticle thermal therapy based on an open magnetic particle image of claim 3, wherein a received signal of the receiver coil is used for measuring the temperature of the nanoparticle.

6. The apparatus for a nanoparticle thermal therapy based on an open magnetic particle image of claim 1, further comprising:
a stage unit for adjusting a position of the target area corresponding to the nanoparticle.

7. The apparatus for a nanoparticle thermal therapy based on an open magnetic particle image of claim 1, wherein the nanoparticle is heated in a first time interval and the temperature of the nanoparticle is measured in a second time interval, and
a third time interval between the first time interval and the second time interval includes a relaxation time.
